(19) United States
(12) Patent Application Publication
(10) Pub. No.: US 2022/0151653 A1
(43) Pub. Date: May 19, 2022

(54) CAP FOR ENDOSCOPE

(71) Applicant: UNITED STATES ENDOSCOPY GROUP, INC., Mentor, OH (US)

(72) Inventors: Reza Mohammadpour, Willoughby Hills, OH (US); Mohamed Lababidi, Olmsted Falls, OH (US); Alex Uspenski, Chardon, OH (US); Cynthia Ann Ranallo, Eastlake, OH (US); Michael Charles Hauser, Chardon, OH (US); Holden Szalek, Mentor, OH (US)

(21) Appl. No.: 17/513,406

(22) Filed: Oct. 28, 2021

Related U.S. Application Data
(60) Provisional application No. 63/106,726, filed on Oct. 28, 2020.

(51) Int. Cl. A61B 17/32; A61B 17/00; A61B 18/14

(52) U.S. Cl. CPC A61B 17/320016; A61B 17/00234; A61B 2018/00601; A61B 2017/00296; A61B 18/1492

(57) ABSTRACT

A device for fragmenting a surgical implant includes a cap. The cap includes a first channel extending from a first end of the cap to the second end of the cap. The device includes a first electrode, a second electrode, and an endoscope coupled with the cap.

### CROSS-REFERENCE TO RELATED APPLICATIONS

The present application claims benefit and priority to U.S. Provisional Patent Application No. 63/106,726, filed on Oct. 28, 2020, the entire disclosure of which is incorporated herein by reference.

### FIELD OF THE INVENTION

The present invention relates to surgical devices and, more particularly, to instruments for endoscopically controlled fragmentation of surgical implants situated in the gastrointestinal tract, in the tracheobronchial system or in other hollow organs.

### BACKGROUND

Flexible endoscopes are axially elongate instruments that can navigate through body lumens of a patient for remotely evaluating and/or treating a variety of ailments. Endoscopes have viewing capability provided by fiber optic elements that transmit images along their length to the medical care provider. Endoscopes may be specifically configured in length, diameter, flexibility, and lumen configuration to navigate to specific treatment areas in the body and conduct specific procedures. Such a specifically configured endoscope may be known by a specific or functional name, for example as a laparoscope, duodenoscope, colonoscope, sigmoidoscope, bronchoscope or ureteroscope.

Polypectomy, or the removal of polyps, is a common endoscopic procedure in gastrointestinal endoscopy. An electrocautery or “hot” snare is often used to remove polyps to reduce the risk of bleeding that can result from the coagulation effect created by the current. For this procedure and other hemostasis or defect closures, it is often necessary to utilize a mechanical clip, staple, or implant in an interventional procedure to prevent or limit bleeding. Such implants can be made of metal or a metallic alloy and are designed to withstand special loads and mechanical cutting tools. Implants are designed to be retained in the body long enough for the treated injury to heal. This can prove to be an issue when an implant needs to be removed from tissue after it has been deployed. There is therefore a fundamental need for an instrument which makes the implant in question easier to remove by fragmentation, melting, or cutting of the implant material, while preventing complications or injury to the tissue surrounding the implant.

### SUMMARY

According to one aspect of the disclosure, a device for fragmenting a surgical implant includes a cap comprising a first end and a second end. In various embodiments, the cap comprises a first channel and a second channel. In various embodiments, the first channel and the second channel extend from the first end of the cap to the second end of the cap. In various embodiments, the first channel receives a first electrode, and the second channel receives a second electrode. In various embodiments, the cap is configured to attach to an endoscope. In various embodiments, the cap comprises one or more protrusions extending from the cap. The protrusions can be spaced apart from the electrodes to allow space for the implant to wedge between them and the electrodes in order to improve the electrical contact between the electrodes and the implant. The protrusions can thus protect the patient and promote electrical connection between the implant clip and electrodes. In various embodiments, the cap is made from a nonconducting material. In various embodiments, the first electrode and the second electrode comprise a bipolar electrode pair. In various embodiments, the device includes a third channel extending from the first end of the cap to the second end of the cap, wherein the third channel is configured to receive an endoscopic instrument. In various embodiments, the device includes a fourth channel extending from the first end of the cap to the second end of the cap, wherein the fourth channel is configured to receive the endoscope. In various embodiments, the device includes a power supply coupled to the first electrode and the second electrode via wiring. In various embodiments, the first electrode and the second electrode receive a direct electric current from the power supply via the wiring. In various embodiments, the first electrode and the second electrode introduce a high-frequency current into the implant. In various embodiments, the implant is separated or distanced from a tissue of a hollow organ during introduction of the high-frequency current from the first electrode and the second electrode to the implant. In various embodiments, the device includes a hood coupled with the cap, wherein the hood is moveable relative to the cap.

According to another aspect of the disclosure, a device for fragmenting a surgical implant includes a cap having a first end and a second end, an endoscope coupled with the cap, a first electrode coupled with the cap, and a second electrode coupled with the cap. In various embodiments, the first electrode and the second electrode comprise a bipolar electrode pair. In various embodiments, the cap comprises a first channel extending from the first end of the cap to the second end of the cap. In various embodiments, the first channel receives the first electrode and the second electrode. In various embodiments, the cap is made from a nonconducting material. In various embodiments, the device includes an endoscopic instrument coupled with the cap. In various embodiments, the endoscope extends through a channel of the cap. In various embodiments, a distal end of at least of the first electrode and the second electrode are positioned proximal to a distal end of the cap. In various embodiments, the first electrode and the second electrode receive a direct electric current from a power supply via a wiring. In various embodiments, the first electrode and the second electrode introduce a high-frequency current into the implant. In various embodiments, the implant is separated or distanced from a tissue of a hollow organ during introduction of the high-frequency current from the first electrode and the second electrode to the implant. In various embodiments, the device includes a hood coupled with the cap and that is moveable relative to the cap.

According to another aspect of the disclosure, a device for fragmenting a surgical implant includes an endoscopic instrument including a first component comprising a first electrode, and a second component coupled with the first component. In various embodiments, the second component is movable along a longitudinal axis of the instrument. In various embodiments, the second component comprises a second electrode. In various embodiments, the distance between the first electrode and the second electrode decreases as the second component moves in a distal direction. In various embodiments, the distance between the first electrode and the second electrode increases as the second component moves in a proximal direction. In various embodiments, the distal end of the first component comprises a nonconducting material.

According to another aspect of the disclosure, a cap includes a first end and a second end. In various embodiments, the cap includes a first channel extending from the first end of the cap to the second end of the cap. the first channel comprises a diameter of 0.065 inches. In various embodiments, the cap includes a second channel extending from the first end of the cap to the second end of the cap. In various embodiments, the second channel comprises a diameter of 0.065 inches. In various embodiments, the cap includes a third channel extending from the first end of the cap to the second end of the cap. In various embodiments, the third channel comprises a diameter of 2.5 mm. In various embodiments, the cap includes a first protrusion extending from the cap in a first direction. In various embodiments, the cap includes a second protrusion extending from the cap in the first direction. In various embodiments, the cap includes a third protrusion extending from the cap in the first direction.

According to another aspect of the disclosure, a device for fragmenting a surgical implant includes a cap comprising a first end and a second end. In various embodiments, the cap comprises a first channel and a second channel. In various embodiments, the first channel and the second channel extend from the first end of the cap to the second end of the cap. In various embodiments, the first channel receives a first fragmentation instrument, and the second channel receives a second fragmentation instrument. In various embodiments, the cap is configured to attach to an endoscope. In various embodiments, the cap comprises one or more protrusions extending from the cap. In various embodiments, the cap is made from a nonconducting material. In various embodiments, the device includes a third channel extending from the first end of the cap to the second end of the cap, wherein the third channel is configured to receive an endoscopic instrument. In various embodiments, the device includes a fourth channel extending from the first end of the cap to the second end of the cap, wherein the fourth channel is configured to receive the endoscope. In various embodiments, the device includes a power supply coupled to the first fragmentation instrument and the second fragmentation instrument via wiring. In various embodiments, the first fragmentation instrument and the second fragmentation instrument receive a direct electric current from the power supply via the wiring. In various embodiments, the first fragmentation instrument and the second fragmentation instrument introduce a high-frequency current into the implant. In various embodiments, the implant is separated or distanced from a tissue of a hollow organ during introduction of the high-frequency current from the first fragmentation instrument and the second fragmentation instrument to the implant. In various embodiments, the device includes a hood coupled with the cap, wherein the hood is moveable relative to the cap.

According to another aspect of the disclosure, a device for fragmenting a surgical implant includes a cap having a first end and a second end, an endoscope coupled with the cap, a first fragmentation instrument coupled with the cap, and a second fragmentation instrument coupled with the cap. In various embodiments, the cap comprises a first channel extending from the first end of the cap to the second end of the cap. In various embodiments, the first channel receives the first fragmentation instrument and the second fragmentation instrument. In various embodiments, the cap is made from a nonconducting material. In various embodiments, the device includes an endoscopic instrument coupled with the cap. In various embodiments, the endoscope extends through a channel of the cap. In various embodiments, a distal end of at least of the first fragmentation instrument and the second fragmentation instrument are positioned proximal to a distal end of the cap. In various embodiments, the first fragmentation instrument and the second fragmentation instrument receive a direct electric current from a power supply via a wiring. In various embodiments, the first fragmentation instrument and the second fragmentation instrument introduce a high-frequency current into the implant. In various embodiments, the implant is separated or distanced from a tissue of a hollow organ during introduction of the high-frequency current from the first fragmentation instrument and the second fragmentation instrument to the implant. In various embodiments, the device includes a hood coupled with the cap and that is moveable relative to the cap.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures can be drawn to scale for some embodiments, the figures are not necessarily drawn to scale for all embodiments. Embodiments and other features and advantages of the present disclosure will be described and explained with additional specificity and detail through the use of the accompanying drawings in which:

FIG. 1 is a perspective view of an exemplary embodiment of a device for fragmenting a surgical implant;

FIG. 2 is an exploded view of a device for fragmenting a surgical implant;

FIGS. 3A-3B are perspective side views of a cap of a device for fragmenting a surgical implant;

FIG. 3C is a perspective front view of a cap of a device for fragmenting a surgical implant;

FIG. 3D is a perspective back view of a cap of a device for fragmenting a surgical implant;

FIG. 4 is a cross sectional view of the cap shown in FIG. 3C, taken along lines A-A;

FIG. 5A is a perspective side view of a cap and electrodes of a device for fragmenting a surgical implant;

FIG. 5B is a cross sectional view of components of a device for fragmenting a surgical implant shown in FIG. 5A, taken along lines B-B;

FIGS. 6A-6B are perspective views of components of a device for fragmenting a surgical implant;

FIG. 7A is a cross sectional view of a fragmentation instrument in accordance with various embodiments;

FIGS. 7B-7C are perspective views of components of a device for fragmenting a surgical implant;

FIG. 8 is a perspective view of an exemplary embodiment of a device for fragmenting a surgical implant;

FIG. 9 is a perspective side view of a cap of a device for fragmenting a surgical implant;

FIGS. 10A-10B are perspective views of components of a device for fragmenting a surgical implant;

FIGS. 11A-11B are perspective views of components of a device for fragmenting a surgical implant;

FIGS. 12A-12B are perspective views of components of a device for fragmenting a surgical implant; and

FIG. 12C is a perspective views from the bottom of a device for fragmenting a surgical implant.

### DETAILED DESCRIPTION

The following description refers to the accompanying drawings, which illustrate specific embodiments of the present disclosure. Other embodiments having different structures and operation do not depart from the scope of the present disclosure.

Exemplary embodiments of the present disclosure are directed to devices and methods for fragmenting a surgical implant. It should be noted that various embodiments of devices and systems for fragmenting a surgical implant are disclosed herein, and any combination of these options can be made unless specifically excluded. In other words, individual components of the disclosed devices and systems can be combined unless mutually exclusive or otherwise physically impossible.

As described herein, when one or more components are described as being connected, joined, affixed, coupled, attached, or otherwise interconnected, such interconnection may be direct as between the components or may be indirect such as through the use of one or more intermediary components. Also, as described herein, reference to a “member,” “component,” or “portion” shall not be limited to a single structural member, component, or element but can include an assembly of components, members, or elements.

Unless otherwise indicated, all numbers such as, for example, numbers or number ranges expressing measurements or physical characteristics, used in the specification and claims are to be understood as being modified in all instances by the term “about.” “Substantially” and “about” are defined as at least close to (and includes) a given value or state (preferably within 10% of, more preferably within 1% of, and most preferably within 0.1% of). Accordingly, unless otherwise indicated, the numerical properties set forth in the specification and claims are approximations that may vary depending on the suitable properties sought to be obtained in embodiments of the invention. Any numerical values, however, inherently contain certain errors necessarily resulting from error found in their respective measurements.

The present application describes various components as being “proximal” or “distal.” As used herein, the term “proximal” refers to a portion of a component that is situated nearer to the center of the body of a device for fragmenting a surgical implant, or to a direction toward the center of the body of the device for fragmenting a surgical implant, unless the context clearly indicates otherwise. As used herein, the term “distal” refers to a portion of a component that is situated away from the center of the body of a device for fragmenting a surgical implant, or to a direction away the center of the body of the device for fragmenting a surgical implant, unless the context clearly indicates otherwise.

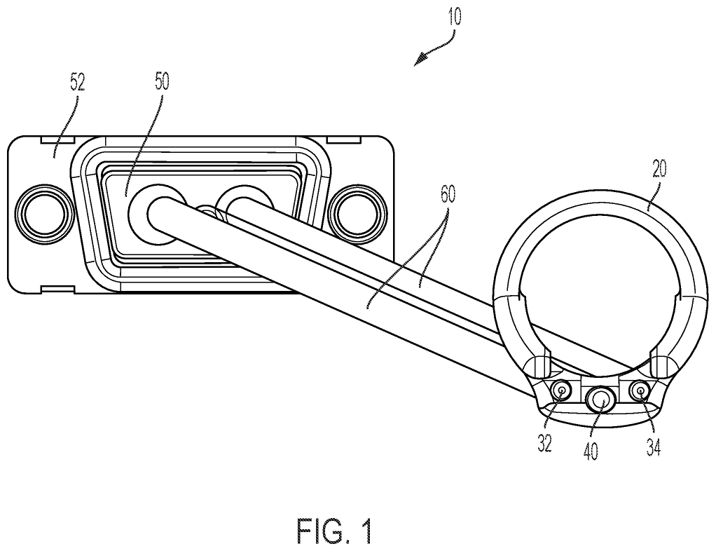

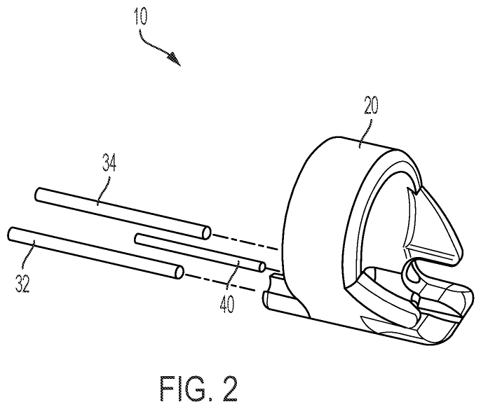

Referring to FIG. 1, illustrated are components of a device 10 for fragmenting a surgical implant and embodiments in accordance with the principles of the present disclosure. The device 10 comprises an endoscope cap, such as, for example, a cap 20, one or more electrodes, for example a first electrode 32 and a second electrode 34, an endoscopic instrument 40, a power supply 50, and a wiring 60 connecting the power supply 50 to the first electrode 32 and second electrode 34. FIG. 2 illustrates an exploded view of various components of the device 10 in accordance with various embodiments of the present disclosure.

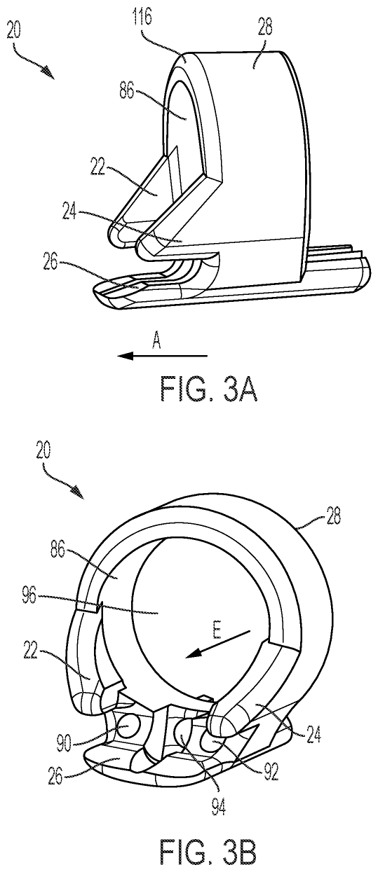

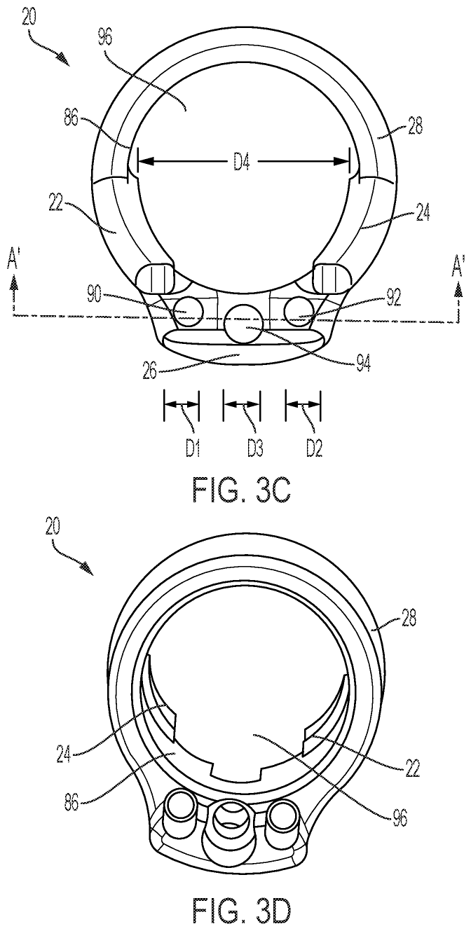

With reference to FIGS. 3A- 3D, a cap 20 is illustrated in accordance with various embodiments. The cap 20 can be made of a flexible material, such as, for example, silicone or a flexible polymer, and is monolithically formed. In some embodiments, the cap 20 comprises a single material. In some embodiments, the cap 20 comprises a combination of materials. In some embodiments, the cap 20 is made of a nonconducting material. In a preferred embodiment, the cap 20 is made of a substantially heat resistant material that can be used in environments of greater than 300-400 degrees F. Materials such as Silicone, High Temperature Resins, Ultem, Vespel (PI or Kapton), Torlon (PAI), PEEK, PEK, PTFE, ARLON, Polyimide, Macor, Ceramics, Epoxy, and Boron Nitride coating are examples of such a material.

In various embodiments, the cap 20 can include one or more protrusions extending from the cap 20. The cap 20 can include, one or more of a first protrusion 22 and a second protrusion 24 extending from the cap 20. The first protrusion 22 and the second protrusion 24 can extend from the cap 20 in a first direction A from the cap 20 (see FIG. 3A). In various embodiments, the cap 20 can include a third protrusion 26 extending from the cap 20. The third protrusion 26 can extend from the cap 20 in the first direction A. The one or more protrusions can be made of the same or a different material as the cap 20. In various embodiments, the one or more protrusions can be made of a nonconducting material.

The protrusions can be made of various shapes and sizes. For example, as shown in FIGS. 3A-3D, the first protrusion 22 and the second protrusion 24 each comprise two surfaces which come to a point at an acute angle. In various embodiments, the protrusions can be rounded or comprise various other shapes.

In various embodiments, the protrusions can be longer than the electrodes in order to protect the patient from the relatively sharp electrodes during insertion and from heating of the electrodes during implant removal. The protrusions are spaced apart from the electrodes (above and below) such that they allow space for the implant to wedge between them and the electrodes in order to improve the electrical contact between the electrodes and the implant. The protrusions can thus protect the patient and promote electrical connection between the implant and electrodes.

An outer surface 28 of the cap 20 can be smooth so as to avoid injuring tissue and other portions of a patient's anatomy as cap 20 is inserted into an internal cavity of the patient. In some embodiments, the surface 28 may have various surface configurations to enhance fixation, such as, for example, rough, arcuate, undulating, porous, semi-porous, dimpled and/or textured, according to the requirements of a particular application.

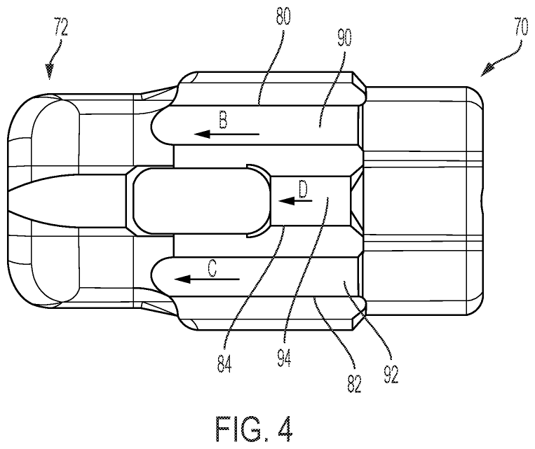

In various embodiments, with reference to FIG. 4, a cross sectional view of the cap 20 of FIG. 3C is shown, taken along lines A′-A′. The cap 20 can include an inner surface 80 extending from a first end 70 of the cap 20 to a second end 72 of the cap 20. Inner surface 80 can define a first channel 90 that extends along a first longitudinal axis B. In various embodiments, the cap 20 can include an inner surface 82 extending from a first end 70 of the cap 20 to a second end 72 of the cap 20, the inner surface 82 defining a second channel 92 that extends along a second longitudinal axis C. In various embodiments, the cap 20 can include an inner surface 84 extending from a first end 70 of the cap 20 to a second end 72 of the cap 20, the inner surface 84 defining a third channel 94 that extends along a third longitudinal axis D.

With reference to FIGS. 3A-D, in various embodiments, the cap 20 can include an inner surface 86 extending from a first end 70 of the cap 20 to a second end 72 of the cap 20, the inner surface 86 defining a fourth channel 96 that extends along a fourth longitudinal axis E (see FIG. 3B).

With reference to FIG. 3C, the first channel 90 can have a diameter D1 ranging from 0.025 inches to 0.125 inches. In various embodiments, first channel 90 preferably has a diameter D1 of 0.065 inches. The second channel 92 can have a diameter D2 ranging from 0.025 inches to 0.125 inches. In various embodiments, the second channel 92 preferably has a diameter D2 of 0.065 inches. The third channel 94 can have a diameter D3 ranging from 1 mm to 5 mm. In various embodiments, the third channel 94 preferably has a diameter D3 of 2.5 mm. The fourth channel 96 can have a diameter D4 ranging from 9 mm to 14 mm. The diameter D4 can depend on the type of endoscope and cap material used.

Channels 90, 92, 94, and 96 can have various cross section configurations, such as, for example, oval, oblong, triangular, rectangular, square, polygonal, irregular, uniform, non-uniform, variable, tubular and/or tapered. In some embodiments, the cross sectional configurations of channels 90, 92, 94, and 96 can be the same or different. In some embodiments, axis B, C, D, and E are parallel. In various embodiments, axes B, C, D, and E may be disposed at alternate orientations, relative to the other axes, such as, for example, transverse, perpendicular and/or other angular orientations such as acute or obtuse, co-axial and/or may be offset or staggered. As shown in FIGS. 3A-3D, axes B, C, D, and E are substantially parallel to one another.

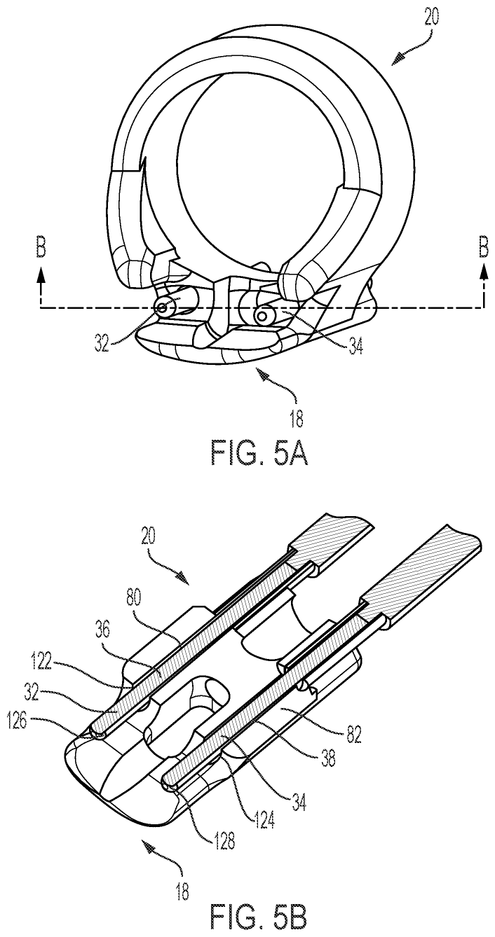

With reference to FIGS. 5A-B the first electrode 32 is disposed in the channel 90 of the cap 20. In some embodiments, the cap 20 is flexible such that it can be stretched to fit the first electrode 32. The first electrode 32 has a diameter that is less than diameter D1 such that an outer surface 36 of the first electrode 32 forms a friction fit with the inner surface 80 of cap 20 when the first electrode 32 is disposed within the channel 90 and coupled with the cap 20.

In various embodiments, the first electrode 32 is disposed in channel 90 such that the first end surface 126 of the first electrode 32 extends beyond the distal surface 122 of channel 90. In various embodiments, the first end surface 126 of the first electrode 32 can be flush with the distal surface 122 of channel 90, or can even be positioned within the channel 90. In various embodiments, the first end surface 126 of the first electrode 32 is positioned between the distal surface 122 of channel 90 and the distal end 18 of the cap 20.

The second electrode 34 is disposed in the channel 92 of cap 20. In some embodiments, the cap 20 flexible such that the cap 20 can be stretched to fit around the second electrode 34. The second electrode 34 has a diameter that is less than diameter D2 such that an outer surface 38 of the second electrode 34 forms a friction fit with the inner surface 82 of cap 20 when the second electrode 34 is disposed within the channel 92 and coupled with the cap 20.

In various embodiments, the second electrode 34 is disposed in channel 92 such that the first end surface 128 of the second electrode 34 extends beyond the distal surface 124 of channel 92. In various embodiments, the first end surface 128 of the second electrode 34 can be flush with the distal surface 124 of channel 92 or even be positioned within the channel 92. In various embodiments, the first end surface 128 of the second electrode 34 is positioned between the distal surface 124 of channel 92 and the distal end 18 of the cap 20.

In some embodiments, the first electrode 32 and second electrode 34 are oriented parallel to one another. In various embodiments, the first electrode 32 and second electrode 34 may be disposed at alternate orientations, relative to the other axes, such as, for example, transverse, perpendicular and/or other angular orientations such as acute or obtuse, co-axial and/or may be offset or staggered. As shown in FIG. 5A, the first electrode 32 and second electrode 34 are oriented parallel to one another.

In various embodiments, the first end surface 126 of the first electrode 32 and the first end surface 128 of the second electrode 34 can extend to be substantially the same length of the one or more of the protrusion (e.g., protrusions 22, 24, 26) of the cap 20. In various embodiments, the first end surface 126 of the first electrode 32 is positioned between the distal surface 122 of channel 90 and the distal end of the one or more of the protrusion (e.g., protrusions 22, 24, 26) of the cap 20. In various embodiments, the first end surface 128 of second electrode 34 is positioned between the distal surface 124 of channel 92 and the distal end of the one or more of the protrusions (e.g., protrusions 22, 24, 26) of the cap 20.

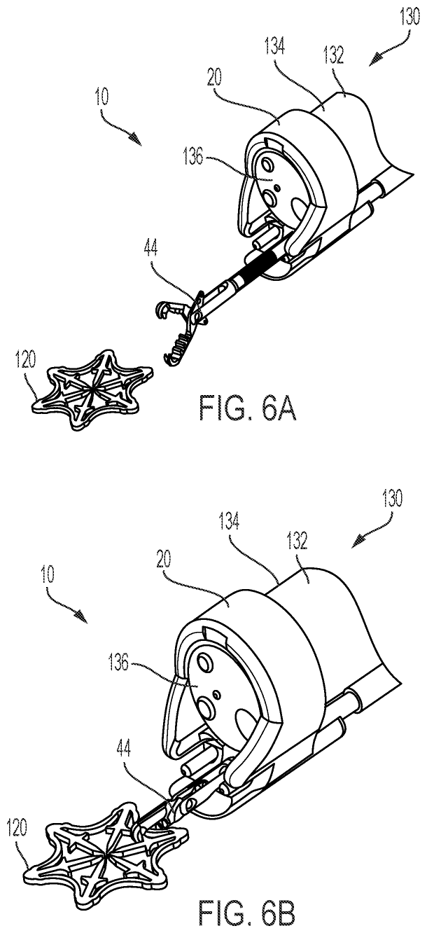

In various embodiments, with reference to FIGS. 6A-6B, the device 10 can include an endoscope 130 comprising a cylindrical shaft 132. In various embodiments, the endoscope 130 can be a laparoscope, duodenoscope, colonoscope, sigmoidoscope, bronchoscope or ureteroscope. In various embodiments, the cylindrical shaft 132 can be disposed in the fourth channel 96. The cylindrical shaft 132 can be inserted through the fourth channel 96 such that the cap 20 fits securely onto the endoscope 130. In embodiments, the cap 20 is configured to be flexible such that cap 20 can be stretched to fit over and secure to the shaft 132. Shaft 132 has a width that is less than diameter D4 such that an outer surface 134 of shaft 132 forms a friction fit with inner surface 86 when the shaft 132 is disposed within channel 96 and couples with the cap 20.

In various embodiments, the shaft 132 is disposed in channel 96 such that a first end surface 136 of shaft 132 is flush with the distal surface 116 of channel 96 (see FIG. 3A). In various embodiments, the shaft 132 can be disposed in channel 96 such that surface 136 extends beyond the distal surface 116 of channel 96 or within the channel 96.

In various embodiments, with reference to FIG. 1, device 10 includes a power source 50 connected to the device 10. The power source 50 can be coupled to the device 10 by connector 52. The power source 50 can comprise a medical DC-impulse generator which is adapted to be connected to the first electrode 32 and the second electrode 34 via wiring 60, or which is connected directly to the first electrode 32 and the second electrode 34. Current can be controlled with a voltage source via stored DC energy in capacitors. The voltage source can create a fixed voltage for a variety of currents. In this manner, a quantity of current, or energy density, is provided in at least one current pulse is sufficient to melt a surgical implant material between the electrodes. In various embodiments, the wiring 60 can extend from the first electrode 32 and the second electrode 34 through the endoscope 130 or an accessory channel of the endoscope 130 towards the power source 50. In various embodiments, the wiring 60 can extend from the first electrode 32 and the second electrode 34 outside of the endoscope 130 towards the power source 50.

By setting the voltage at a predetermined amount, and by measuring the approximate resistance of the human body and the implant, a sufficient current can be determined such that the application of energy does not harm the patient during performance of the fragmenting process. Further, pulsing the DC energy significantly reduces the current load on the implant and patient and therefore reduces the heat generated by the implant during fragmentation. The medical direct current generator preferably has the CPU or is connected to a CPU or another control device of this kind, for example analogue control circuit. The CPU can be adapted to determine and control the electric current flowing through the electrodes such that the performance of the fragmenting process is implemented.

The power source 50 is designed to send an electrical direct current through the first electrode 32 and the second electrode 34. This DC pulse flows through the surgical implant, wherein the first electrode 32 and second electrode 34 at the distal tip of the device 10 are establishing physical contact with the implant 120 (see also FIGS. 6 A-B), resulting in localized heating and melting of the implant 120, resulting in the fracturing of the implant 120. The power source 50 delivers a pulse of preferably optionally between 15-40 volts, including between 20-25 volts. The current delivered through the implant is between 40-60 Amps, including between 45-50 Amps.

In various embodiments, the voltage or the pulse width can be adjusted to selectively break one side or multiple sides of the implant. A pulse width of 200 ms and voltage of 20 V is generally sufficient for breaking one link or portion on the implant, whereas increasing the pulse width to at least 300-400 ms can allow for breaking multiple links or portions of the implant. Higher voltages can be also used to promote faster heating and subsequent fracture of the implant. The lowest voltage that can be used to achieve can be preferable in certain instances to reduce excessive heating of surrounding tissue. Fracturing multiple portions of the implant in one pulse can be advantageous because you can remove the implant with one shot of energy. Extra energy may be required to do this, resulting in additional heat being delivered into the surrounding tissue. In various embodiments, the user can select a “single point” cutting setting vs a “multiple point” cutting setting.

In order to avoid tissue damage, the one or more protrusions extending from the cap 20 (e.g., first protrusion 22, a second protrusion 24, and/or third protrusion 26) serve to space the implant 120 from the tissue of the patient against which it lies or by which it is surrounded, thereby protecting the tissue from damage by an electrically charged electrode. For this purpose, the cap 20 can be formed from heat-resistant and arc-resistant material and can therefore be electrically insulating. By this means, an implant 120 can be separated from the tissue in a simple manner in order to prevent tissue from coming between the implant and the electrode. In various embodiments, the one or more protrusions act as a guide configured such that when the cap 20 is pressed against the tissue or the implant, the implant can be positioned between the one or more protrusions and the electrodes. Surrounding tissue can thereby be protected when localized heating and melting of the implant 120, resulting in the fracturing of the implant 120.

The one or more electrodes can connect to the implant 120 at separate points along the implant's length, delivering energy between them instead of at a distinct point on the implant 120. This allows for cutting of portions of the implant 120 that are not easily accessible with endoscopes and is enabled by the size of the conductors we can use by routing them outside the scope, by, for example, 18 Ga-12 Ga insulated copper, including 14 Ga insulated copper.

In various embodiments, with reference to FIGS. 1-2, the device 10 can include an endoscopic instrument 40 coupled with the cap 20. The endoscopic instrument 40 can be disposed in channel 94 of cap 20. In some embodiments, cap 20 flexible such that cap 20 can be stretched to fit the endoscopic instrument 40. The endoscopic instrument 40 has a diameter that is less than diameter D3 (see FIG. 3C) such that the endoscopic instrument 40 can be moved within the inner surface 84 of cap 20 when endoscopic instrument 40 is moved within channel 94.

In various embodiments, with reference to FIGS. 6A-B, the endoscopic instrument 40 can comprise a forceps device or a grasping device 44. The device 44 can be rotatable to allow for easier positioning relative to the implant 120. With reference to FIGS. 6A-B, after the implant 120 is fractured, the device 44 can be used to grasp and safely remove the implant 120 from the internal cavity of the patient.

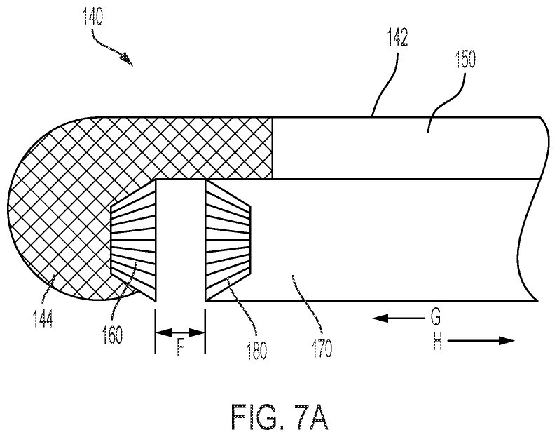

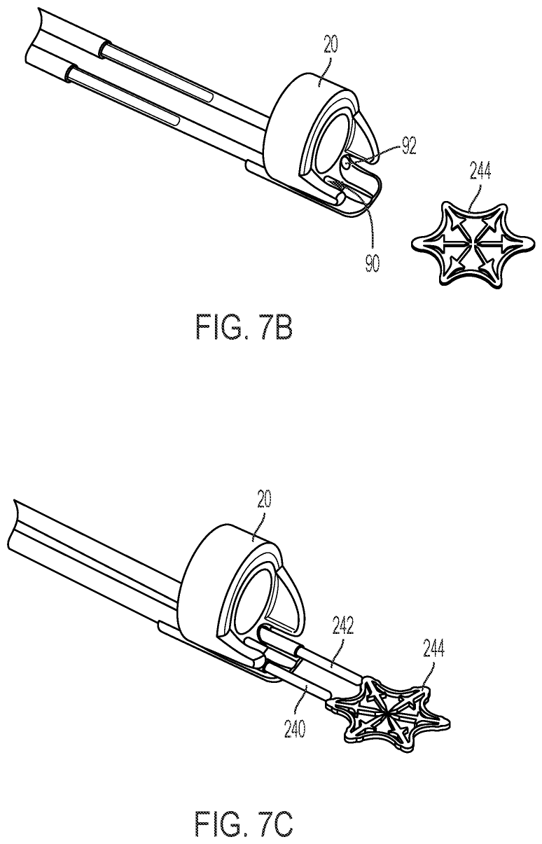

With reference to FIG. 7A, the endoscopic instrument 40 can comprise a fragmentation instrument 140. In various embodiments, the fragmentation instrument 140 can be disposed in first channel 90, second channel 92, or third channel 94 of cap 20 (see FIGS. 3A-D) or within the accessory channel of the endoscope 132.

In some embodiments, cap 20 can be flexible such that cap 20 can be stretched to fit the fragmentation instrument 140. The fragmentation instrument 140 has a diameter that is less than diameter D3 such that an outer surface 142 of the fragmentation instrument 140 can be moved within the inner surface 84 of cap 20 when the fragmentation instrument 140 is disposed within third channel 94. In various embodiments, the diameter of the fragmentation instrument 140 can be from about 2.0 mm to about 3.0 mm. In various embodiments, the diameter of the fragmentation instrument 140 is preferably 2.5-2.6 mm. In various embodiments, fragmentation instrument 140 has a distal end 144 of the instrument comprises a nonconducting material.

With reference to FIG. 7A, the fragmentation instrument 140 comprises a bipolar electrode pair, however in various embodiments, the electrodes in the fragmentation instrument 140 can be monopolar. In various embodiments, the fragmentation instrument 140 comprises a first component 150. In various embodiments, the first component 150 comprises a first electrode 160. In various embodiments, the fragmentation instrument 140 comprises a second component 170 coupled with the first component 150. The second component 170 can be movable along the fragmentation instrument 140. In various embodiments, a distal end 172 of the second component 170 comprises a second electrode 180. In various embodiments, the entire fragmentation instrument 140 may represent one pole of a bipolar pair disposed within channel 90 or 92. In such embodiments, one fragmentation instrument 140 may be electrically connected to one segment of implant 120 while a second fragmentation instrument 140 may be electrically connected to another segment of implant 120, thereby completing the circuit. Such an embodiment may prove advantageous by increasing the size of conductors used to cut the implant 120 and by allowing for extension and retraction of the electrodes or fragmentation instruments from the cap. It can be understood by one skilled in the art that various embodiments of the probe tips, such as a grasper or prongs equally spaced from one another around a central axis, may be employed to achieve a similar effect.

In various embodiments, the distance F between the first electrode 160 and the second electrode 180 decreases as the second component 170 moves in a first direction G. The distance between the first electrode 160 and the second electrode 180 increases as the second component moves in a second direction H.

In various embodiments, the fragmentation instrument 140 is coupled to a power source (e.g., power source 50). The power source preferably contains a current source which is adapted to apply a direct current of predetermined or adjustable strength (current value in ampere) in a pulsed or timed way to the first electrode 160 and the second electrode 180. In this manner, a quantity of current, or energy density, is provided in at least one current pulse is sufficient to melt a surgical implant material between the electrodes. The medical direct current generator preferably has the CPU or is connected to a CPU or another control device of this kind, for example analogue control circuit. The CPU can be adapted to determine and control the electric current flowing through the electrodes such that the performance of the fragmenting process is implemented.

In various embodiments, the implant 120 (see FIGS. 6A-B) can be first positioned between the first component 150 and the second component 170. The second component 170 can be moved towards the distal end 144 of the first component 150 until there is contact between the implant and both the first electrode 160 and the second electrode 180.

Thereafter, the power source can send an electrical direct current through the electrodes and to the surgical implant. This can result in localized heating and melting of the implant and subsequent fracturing of the implant. The power source 50 delivers a direct pulse of preferably optionally between 15-40 volts.

In order to avoid tissue damage, the distal end 144 of the fragmentation instrument 140 can space the implant 120 from the tissue of the patient against which it lies or by which it is surrounded, thereby protecting the tissue from damage by an electrically charged electrode. For this purpose, the fragmentation instrument 140 can be formed from heat-resistant and arc-resistant material and can therefore be electrically insulating. By this means, an implant 120 can be separated from the tissue in simple manner in order to prevent tissue from coming into contact with the electrodes. In various embodiments, the distal end 144 of the fragmentation instrument 140 can act as a guide such that when the fragmentation instrument 140 is pressed against the tissue near the implant 120, the implant 120 can be positioned between the first electrode 160 and the second electrode 180.

In various embodiments, the cap 20 can include two or more fragmentation instruments 140 extending through the channels of the cap. In various embodiments, the two or more fragmentation instruments 140 can be moved through their respective channels of the cap independent from each other. The two or more fragmentation instruments 140 can attach to the implant 120 at different points of the implant 120.

With reference to FIGS. 7B-C, the cap 20 includes a first fragmentation instrument 240 extending through the first channel 90, and a second fragmentation instrument 242 extending through the second channel 92. The first fragmentation instrument 240 and the second fragmentation instrument 242 can be identical to the fragmentation instrument 140 (FIG. 7A) in material aspects.

With reference to FIG. 7C, the first fragmentation instrument 240 and the second fragmentation instrument 242 can comprise monopolar electrodes. The first fragmentation instrument 240 and the second fragmentation instrument 242 can be moved through their respective channels of the cap independent from each other. The first fragmentation instrument 240 can be electrically connected to one segment of implant 244 while the second fragmentation instrument 242 can be electrically connected to another segment of implant 244. Such an embodiment may prove advantageous by allowing for extension and retraction of the fragmentation instruments from the cap. It can be understood by one skilled in the art that various embodiments of the probe tips, such as a grasper or prongs equally spaced from one another around a central axis, may be employed to achieve a similar effect. For each fragmentation instrument, with reference to FIG. 7A, the second component 170 can be moved towards the distal end 144 of the first component 150 until there is contact between the implant 244 (FIG. 7C) and both the first electrode 160 and the second electrode 180. Thereafter, the power source can send an electrical direct current through the electrodes and to the implant 244. This can result in localized heating and melting of the implant 244 and subsequent fracturing of the implant 244. The power source delivers a direct pulse of preferably optionally between 15-40 volts.

In various embodiments, the cap 20 can include a fragmentation instruments 140 extending through the third channel 94, and a second fragmentation instrument 140 extending through one of the first channel 90, the second channel 92, or an accessory channel of the endoscope 132 (see FIG. 6A-B). In various embodiments, the cap 20 can include three fragmentation instruments 140, with one extending through each of the first channel 90, second channel 92, and the third channel 94.

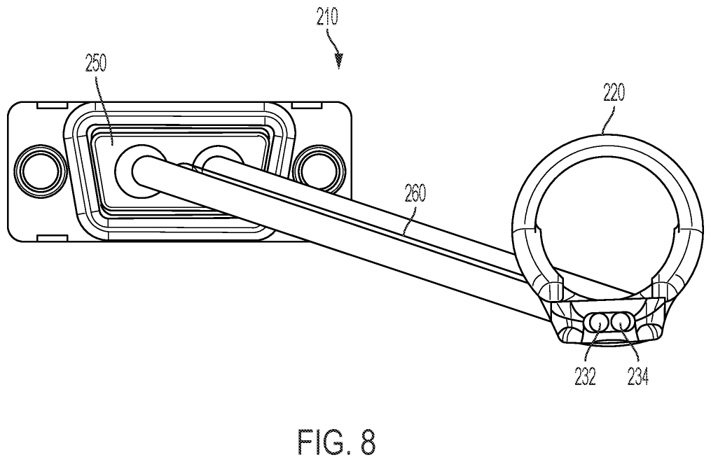

With reference to FIG. 8, in various embodiments, device 210 comprises an endoscope cap, such as, for example, a cap 220, one or more electrodes 232, 234, a power supply 250, and wiring 260 connecting the power supply 250 to the first electrode 232 and the second electrode 234. Cap 220 can be made of a flexible material and can be made of the same material as the cap 20. For example, cap 220 can be made of silicone or a flexible polymer, and be monolithically formed. In some embodiments, the cap 220 comprises a single material. In some embodiments, the cap 220 comprises a combination of materials. In some embodiments, the cap 220 is made of a nonconducting material.

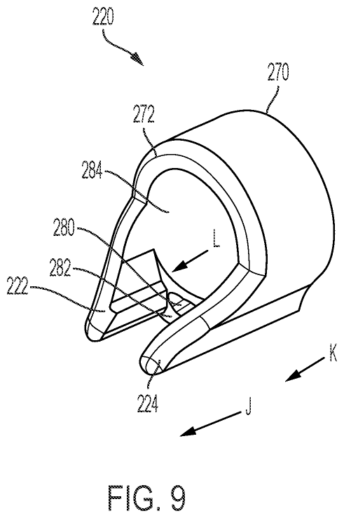

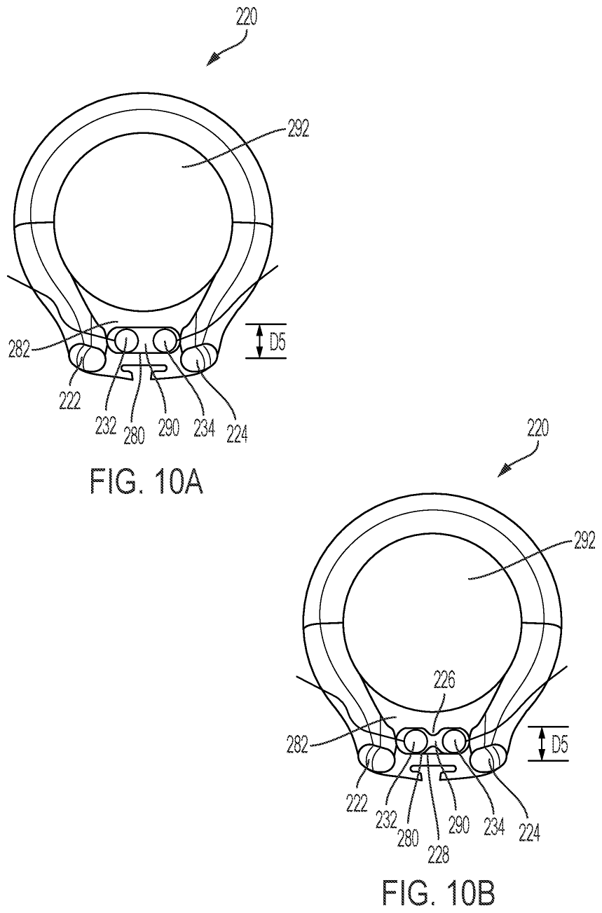

In various embodiments, with reference to FIGS. 9 and 10A-B, the cap 220 can include one or more protrusions extending from the cap 220. The cap 220 can include, one or more of a first protrusion 222 and a second protrusion 224 extending from the cap 20. With reference to FIG. 9, the first protrusion 222 and second protrusion 224 can extend from the cap 220 in a first direction J away from the cap 220. In various embodiments, the one or more protrusions can be made of a nonconducting material. The protrusions can be made of various shapes and sizes. For example, the first protrusion 222 and the second protrusion 224 each comprise two surfaces which come to a point. In various embodiments, the protrusions can be rounded or comprise various other shapes.

With reference to FIG. 8, device 210 comprises a bipolar pair of electrodes. The device 210 can include a first electrode 232 and a second electrode 234. It is envisioned that the shapes and sizes of the electrodes can be selected to provide a desired result during a procedure. In various embodiments, the first electrode 232 and the second electrode 234 are the same size and shape and are otherwise identical in nature. With reference to FIGS. 8-10B, in various embodiments, at least one of the first electrode 232 and the second electrode 234 can be replaced with monopolar fragmentation instruments 140 (see FIG. 7A).

With reference to FIG. 9, the cap 220 can include an inner surface 280 extending from a first end 270 of the cap 220 to a second end 272 of the cap 220. Inner surface 280 can define a first channel 290 (see FIGS. 10A-B) that extends along a longitudinal axis K. The channel 290 can have a width D5 of 0.050 inches to 0.25 inches. The channel 290 can preferably have a width D5 of X. With reference to FIG. 10B, the cap 220 can optionally comprise one or more protrusions (226, 228) extending from inner surface 280 between the first electrode 232 and the second electrode 234.

In various embodiments, the first electrode 232 and second electrode 234 are disposed in channel 290. First electrode 232 has a diameter that is less than width D5 such that an outer surface of first electrode 232 forms a friction fit with the inner surface 280 of cap 220 when the first electrode 232 is disposed within channel 290 and coupled with the cap 220. Second electrode 234 also has a diameter that is less than width D5 such that an outer surface of second electrode 234 forms a friction fit with the inner surface 280 of cap 220 when the second electrode 234 is disposed within channel 290 and coupled with the cap 220.

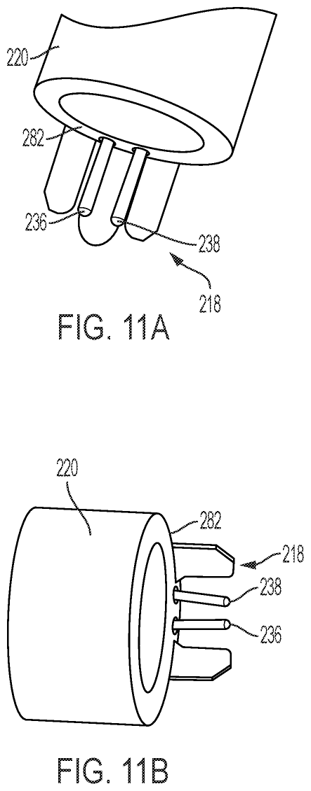

With reference to FIGS. 11A-B, the first end surface 236 of the first electrode 232 extends beyond the distal surface 282 of channel 290. The first end surface 236 of the first electrode 232 is positioned between the distal surface 282 of channel 290 and the distal end 218 of the cap 220. The first end surface 238 of the second electrode 234 also extends beyond the distal surface 282 of channel 290. The first end surface 238 of the second electrode 234 is positioned between the distal surface 282 of channel 290 and the distal end 218 of the cap 220. In various embodiments, the first end surface 236 of the first electrode 232 and the first end surface 238 of the second electrode 234 can extend to be the same length of the one or more protrusion (e.g., protrusions 222, 224) of the cap 220.

In some embodiments, the first electrode 232 and second electrode 234 are oriented parallel to one another. In various embodiments, the first electrode 232 and second electrode 234 may be disposed at alternate orientations, relative to the other axes, such as, for example, transverse, perpendicular and/or other angular orientations such as acute or obtuse, co-axial and/or may be offset or staggered. As shown in FIG. 11A, the first electrode 232 and second electrode 234 are oriented parallel to one another. As shown in FIG. 11B, the first electrode 232 and second electrode 234 are oriented towards one another.

In various embodiments, with reference to FIG. 9, the cap 220 can include an inner surface 284 extending from a first end 270 of the cap 220 to a second end 272 of the cap 220, the inner surface 284 defining a second channel 292 (see FIGS. 10A-B) that extends along a second longitudinal axis L. Channels 290, 292 can have various cross section configurations, such as, for example, oval, oblong, triangular, rectangular, square, polygonal, irregular, uniform, non-uniform, variable, tubular and/or tapered. In various embodiments, the device 210 can include an endoscopic device (e.g., endoscope 130) in the channel 292 of the cap 220 of device 10.

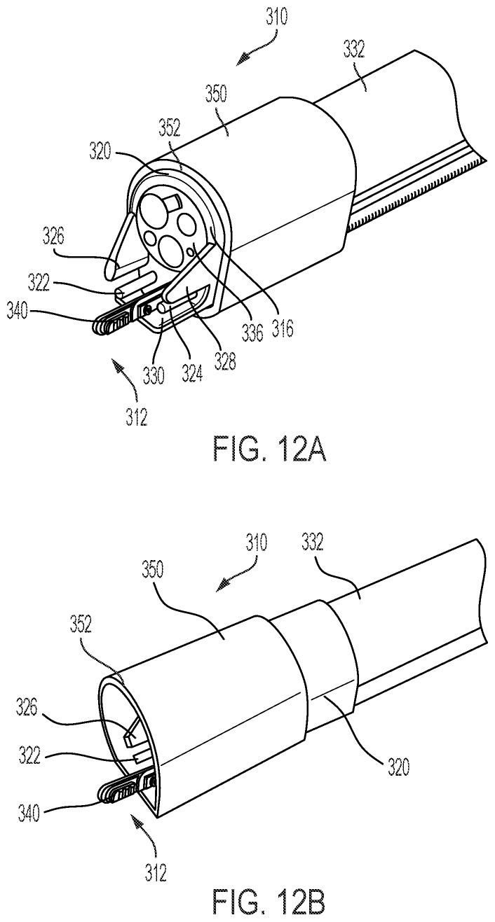

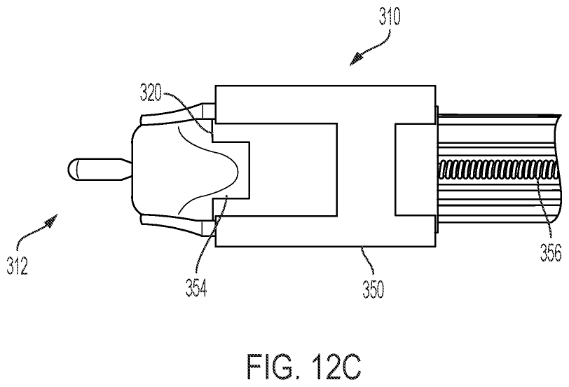

In various embodiments, with reference to FIGS. 12A-C, the device 310 for fragmenting a surgical implant can include a hood 350 that can be moved over the distal end 312 of the device 310. The hood 350 can have a retracted position (FIG. 12A), wherein the distal end 352 of the hood 350 is flush with or proximal to the first end surface 336 of shaft 332 or the distal surface 316 of channel 96 (see FIG. 3A).

The reference to FIG. 12B, the hood 350 can be extended towards the distal end 312 of the device 310 such that the distal end 352 of the hood 350 extends over or past the distal end of at least one of the first electrode 322, the second electrode 324, one or more of the protrusions extending from the cap 320 (e.g., first protrusion 326, a second protrusion 328, and/or third protrusion 330), or the endoscopic instrument 340. This position comprises an “extended position” of the hood 350. In various embodiments, the hood 350 can extend to protect the patient from the electrodes or fragments of the implant as the fragments are withdrawn from the patient. In various embodiments, at least one of the first electrode 322 and the second electrode 324 can be replaced with monopolar fragmentation instruments 140 (see FIG. 7A).

With reference to FIG. 12C, the device 310 can include a stop 354 which can make contact with the hood 350 at the extended position to prevent the hood 350 from moving further towards the distal end 312 of the cap 320. The stop 354 can be coupled with or integral with the cap 320.

Any structure that can allow the hood 350 to move proximally or distally relative to the cap 320 can be used. In one exemplary embodiment, with reference to FIG. 12C, the hood 350 can be coupled with a drive cable 356. The drive cable 356 can push or pull the cap 320 and/or hood 350 to reposition the hood 350 relative to the cap 320.

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

1. A device for fragmenting a surgical implant, the device comprising:
a cap comprising a first end and a second end;
wherein the cap comprises a first channel and a second channel, the first channel and the second channel extending from the first end of the cap to the second end of the cap,
wherein the first channel receives a first electrode, and wherein the second channel receives a second electrode, wherein the cap is configured to attach to an endoscope.

2. The device of claim 1, wherein the cap comprises one or more protrusions extending from the cap.

3. The device of claim 1, wherein the cap is made from a nonconducting material.

4. The device of claim 1, wherein the first electrode and the second electrode comprise a bipolar electrode pair.

5. The device of claim 1, further comprising a third channel extending from the first end of the cap to the second end of the cap, wherein the third channel is configured to receive an endoscopic instrument.

6. The device of claim 5, further comprising a fourth channel extending from the first end of the cap to the second end of the cap, wherein the fourth channel is configured to receive the endoscope.

7. The device of claim 1, further comprising a power supply coupled to the first electrode and the second electrode via wiring.

8. The device of claim 7, wherein the first electrode and the second electrode receive a direct electric current from the power supply via the wiring, wherein the first electrode and the second electrode introduce a high-frequency current into the implant, wherein the implant is separated or distanced from a tissue of a hollow organ during introduction of the high-frequency current from the first electrode and the second electrode to the implant.

9. The device of claim 1, further comprising a hood coupled with the cap, wherein the hood is moveable relative to the cap.

10. A device for fragmenting a surgical implant, the device comprising:
a cap comprising a first end and a second end,
an endoscope coupled with the cap;
a first electrode coupled with the cap; and
a second electrode coupled with the cap.

11. The device of claim 10, wherein the first electrode and the second electrode comprise a bipolar electrode pair.

12. The device of claim 10, wherein the cap comprises a first channel extending from the first end of the cap to the second end of the cap, wherein the first channel receives the first electrode and the second electrode.

13. The device of claim 10, wherein the cap comprises one or more protrusions extending from the cap.

14. The device of claim 10, wherein the cap is made from a nonconducting material.

15. The device of claim 10, further comprising an endoscopic instrument coupled with the cap.

16. The device of claim 10, wherein the endoscope is disposed in a channel of the cap.

17. The device of claim 10, wherein a distal end of at least of the first electrode and the second electrode are positioned proximal to a distal end of the cap.

18. The device of claim 17, wherein the first electrode and the second electrode receive a direct electric current from a power supply via a wiring, wherein the first electrode and the second electrode introduce a high-frequency current into the implant, wherein the implant is separated or distanced from a tissue of a hollow organ during introduction of the high-frequency current from the first electrode and the second electrode to the implant.

19. The device of claim 10, further comprising a hood coupled with the cap, wherein the hood is moveable relative to the cap.

20-41. (canceled)

42. A device for fragmenting a surgical implant, the device comprising:
a cap comprising a first end and a second end,
an endoscope coupled with the cap;
a first fragmentation instrument coupled with the cap; and
a second fragmentation instrument coupled with the cap.

43-50. (canceled)
